SEQUENCE LISTING (24 sequences)

SEQ ID NO: 1 - DNA, 26 bp, Homo sapiens
gaagagtgcgatcaagaacccatgac

SEQ ID NO: 2 - DNA, 27 bp, Homo sapiens
gtctctcctccttctcctcctatcttt

SEQ ID NO: 3 - DNA, 24 bp, Homo sapiens
tgctgaaacattcaccttccatgc

SEQ ID NO: 4 - DNA, 22 bp, Homo sapiens
ttcacgagctcaacaagtgcag

SEQ ID NO: 5 - DNA, 23 bp, Homo sapiens
acccgaactttccaagccataac

SEQ ID NO: 6 - DNA, 22 bp, Homo sapiens
tccagcacatctcctctgcaac

SEQ ID NO: 7 - DNA, 24 bp, Homo sapiens
taccgcactcttgcagaaggacaa

SEQ ID NO: 8 - DNA, 26 bp, Homo sapiens
tgcacgtctttgactccttgaaaccc

SEQ ID NO: 9 - DNA, 24 bp, Homo sapiens
tgaaggatgaggccgtctgggaga

SEQ ID NO: 10 - DNA, 24 bp, Homo sapiens
cagtgggcaccgagaagctgaagt

SEQ ID NO: 11 - DNA, 21 bp, Homo sapiens
gcactcctcacaggactcttg

SEQ ID NO: 12 - DNA, 20 bp, Homo sapiens
cccaggtgtaccgtgaagac

SEQ ID NO: 13 - DNA, 25 bp, Homo sapiens
gtgaccaaatcagtgtgaggaggta

SEQ ID NO: 14 - DNA, 25 bp, Homo sapiens
aggaggagttaatggtgctaactgg

SEQ ID NO: 15 - DNA, 20 bp, Homo sapiens
cttcgctacctgcctaaccc

SEQ ID NO: 16 - DNA, 21 bp, Homo sapiens
gactgtgtcaaatcctgctcc

SEQ ID NO: 17 - DNA, 22 bp, Homo sapiens
ctacagataggtattaaggaca

SEQ ID NO: 18 - DNA, 23 bp, Homo sapiens
gcttcatatccatgcagcaccac

SEQ ID NO: 19 - DNA, 20 bp, Homo sapiens
aacgtaattgcatcagccct

SEQ ID NO: 20 - DNA, 20 bp, Homo sapiens
ggtcattctggggtatccac

SEQ ID NO: 21 - DNA, 20 bp, Homo sapiens
gttttctctggggtcgatga

SEQ ID NO: 22 - DNA, 20 bp, Homo sapiens
atttggcttcttgccatcaa

SEQ ID NO: 23 - DNA, 22 bp, Homo sapiens
ttcaatcatggaccaaaatcaa

SEQ ID NO: 24 - DNA, 20 bp, Homo sapiens
tgagtgacagagctgccaag

(19) United States
(12) Patent Application Publication
(10) Pub. No.: US 2016/0256672 A1
(43) Pub. Date: Sep. 8, 2016

(54) INDUCED PLURIPOTENT STEM CELL-DERIVED HEPATOCYTE BASED BIOARTIFICIAL LIVER DEVICE

(71) Applicant: CEDARS-SINAI MEDICAL CENTER, Los Angeles, CA (US)

(72) Inventors: Vaithilingaraja Arumugaswami, Los Angeles, CA (US); Clive Svendsen, Pacific Palisades, CA (US)

(73) Assignee: CEDARS-SINAI MEDICAL CENTER, Los Angeles, CA (US)

(21) Appl. No.: 15/019,938

(22) Filed: Feb. 9, 2016

Related U.S. Application Data
(60) Provisional application No. 62/114,245, filed on Feb. 10, 2015.

(51) Int. Cl. A61M 37/00; A61M 1/16; C12N 5/071

(52) U.S. Cl. CPC A61M 37/00; C12N 5/067; A61M 1/1698; C12N 2506/45; C12N 2533/52; C12N 2501/16; C12N 2501/165; C12N 2533/70; C12N 2501/115; C12N 2501/155; C12N 2501/11; C12N 2501/148; C12N 2501/12; A61M 2205/04; A61M 2202/09

(57) ABSTRACT

Human induced pluripotent stem cell (iPSC) technology combined with a hollow fiber based bioartificial liver (BAL) device can benefit patients with liver failure. Defined iPSC lines can provide unlimited supply of functional hepatocytes by developing iPSC derived hepatocytes (iHeps). Disclosed herein is a protocol for deriving metabolically active hepatocytes from iPSCs. In some embodiments, iHeps were cultured on microcarrier beads in spinner flasks. Subsequently, the iHep-microcarrier complexes were loaded into the extracapillary space of a hollow fiber bioreactor cartridge and cultured using closed circuit continuous flow system. The iHeps secreted human albumin, prothrombin and apolipoprotein B into the hollow fiber intracapillary space media which indicated the maintenance of plasma protein secretory function. In addition, the continuous flow system improved the maturation of iHeps. Thus, the iPSC hepatocytes in the bioartificial liver device maintained the secretory function and exhibited cell maturation.

### CROSS-REFERENCE TO RELATED APPLICATIONS

The application claims priority under 35 U.S.C. §119 to U.S. Provisional Application Ser. No. 62/114,425, entitled “Induced Pluripotent Stem Cell-Derived Hepatocyte Based Bioartificial Liver Device,” by Vaithilingaraja Arumugaswami, et al., filed Feb. 10, 2015, the disclosure of which is incorporated in its entirety by this reference.

### FIELD OF THE INVENTION

The claimed invention relates to therapeutic devices, methods, and applications for patients with acute or chronic liver failure.

### BACKGROUND

According to the U.S. Centers for Disease Control, an estimated 500 million people worldwide are infected with viral hepatitis, and an estimated one million people die annually from related causes. Many other autoimmune and toxic exposures can also lead to end-stage liver disease: 35% of heavy drinkers develop acute alcoholic hepatitis and drugs as common as acetaminophen can lead to acute liver injury. Patients with acute liver failure (ALF) have a mortality of over 80%. Both acute and chronic liver disease can lead to liver failure and require costly transplantation.

An extra-corporeal bioartificial liver (SAL) can extend the life span of ailing patients waiting for a donor organ and also allow the injured liver to regenerate, thus obviating the need for transplantation in some cases. Acute liver failure (ALF) due to viral hepatitis, acetaminophen poisoning, and exacerbation of chronic liver disease may have a fatality rate of over 80%. During liver failure, building up of toxic compounds can lead to brain damage and multiple organ failure. Therefore, liver failure generally requires liver transplantation. However, the limited supply of donors limits the availability of transplantation.

### BRIEF DESCRIPTION OF THE FIGURES

The patent or application file contains at least one drawing executed in color. Copies of this patent or patent application publication with color drawing(s) will be provided by the Office upon request and payment of the necessary fee. Exemplary embodiments are illustrated in referenced figures. It is intended that the embodiments and figures disclosed herein are to be considered illustrative rather than restrictive.

FIG. 1 depicts, in accordance with various embodiments of the present invention, a schematic illustration of the various steps involved in preparing a bioartificial liver device with induced pluripotent stem cell (iPSC) hepatocytes. The illustrated protocol shows that the iPSCs are expanded and differentiated into hepatocytes. In some embodiments, the iPSC-derived hepatocytes are loaded in the bioreactor for preclinical functional evaluation.

FIGS. 2A-2F depict, in accordance with various embodiments of the present invention, results demonstrating the Differentiation of iPSCs into hepatic lineage cells. (A) FIG. 2A is a schematic diagram showing an experimental outline of differentiation of human iPSC into functional hepatocytes. (B) FIG. 2B shows the immunocytochemistry of endoderm marker expression at day 4 differentiation (scale bar 50 μm). (C) FIG. 2C shows the flow cytometry analysis of endoderm marker expression at day 5 differentiation. AFP and albumin (ALB) producing cell population at day 15 and day 21 of differentiation. (D, E and F) Phase I, II and III CYP gene expression analysis. Relative gene expression to undifferentiated iPSC was calculated for iHeps, HepG2 and primary human hepatocytes (PHH) and presented in the bar graphs with standard deviations.

FIGS. 3A-3D depict, in accordance with various embodiments of the present invention, various graphs illustrating results of experimentation showing functional and morphological analysis of hepatic cells derived from iPSC. (A) FIG. 3A shows induction of CYP34A mRNA expression by rifampicin stimulation on day 20 of hepatic lineage cells derived from H9 ES and iPS cells. Housekeeping gene (PPIG) normalized CYP3A4 mRNA level of unstimulated cells was used for calculating fold induction. (B) FIG. 3B shows PAS staining for glycogen storage in iPSC derived hepatocytes (scale bar 10 μm). Liver cancer cell line Huh7.5.1 is included as a control. (C) FIG. 3C shows bright field microscopic image of the polygonal morphology of iPSC derived hepatocytes with defined tight junctions at day 21 post differentiation (scale bar 10 μm). (D) FIG. 3D shows ultrastructural analysis of iPSC derived hepatocytes (day 15) which reveals formation of hepatic features like tight junction (TJ; arrows) and biliary canaliculi (BC) between two adjacent cells (scale bar 0.25 μm). The cells were grown as two dimensional monolayer culture before processing for electron microscopic study. (N: nucleus; C: cytoplasm).

FIGS. 4A-4B depict, in accordance with various embodiments of the present invention, suspension culture of iHeps on dextran microcarrier beads. (A) FIG. 4A is a perspective view of an example of iPSC hepatocytes culture in spinner flask in the context of cell culture incubator. (B) FIG. 4B is an example illustration of bright field and fluorescent images of hepatocytes attached onto dextran microcarrier spheres (scale bar 50 μm). For nuclear staining, cells containing beads were incubated with Hoechst dye for 10 minutes.

FIGS. 5A-5B depict, in accordance with various embodiments of the present invention, an example closed circuit hollow fiber bioreactor system for culturing iPSC-hepatocytes. (A) FIG. 5A shows a schematic diagram displaying the various components of HFB culture setup. The membranous hollow fibers are encased in a cartridge and each fiber is connected to vestibules at the inflow and outflow ends. The functional cells adhere to the outside of each fiber, thus mimicking the architecture of blood capillary and the surrounding parenchyma cells. The hollow fiber contains numerous micropores that allow the exchange of metabolites and proteins. The cells are bathed in continuous flow of oxygenated growth media pumped from a media reservoir. (B) FIG. 5B shows the closed circuit continuous flow setup including hollow fiber bioreactor (HFB) cartridge loaded with iPSC hepatocytes inside a 37° C. incubator. The iPSC hepatocyte loaded HFB cartridge may function as a bioartificial liver device. P: pump; O: membrane oxygenator; M: media bag inside the tray.

FIGS. 6A-6E depict, in accordance with various embodiments of the present invention, graphs from experimental results illustrating a sample functional assessment of iHeps cultured in the hollow fiber bioreactor cartridge with continuous flow. FIGS. 6A-6D show plasma proteins albumin, apolipoprotein B, and prothrombin, and glucose secretion by iHeps at the indicated time points. The mean values with standard deviations are presented in the graphs. FIG. 6E shows relative gene expression of input day 0 iPSC hepatocytes in the graphs with standard deviations. Note the significant reduction of AFP level in cells cultured on continuous flow condition. 2D: two dimensional culturing in dish; BR: bioreactor culturing.

FIG. 7 depicts, in accordance with various embodiments of the present invention, a diagram of an example iHep-based extra-corporeal closed circuit BAL system for supporting patients with liver failure. The BAL system that may be implemented using iHeps. In some embodiments, plasma separated from a patient's blood will be passed through a hollow fiber cartridge containing iHeps that will remove toxic metabolites.

FIG. 8 depicts, in accordance with various embodiments of the present invention, a table of primers that may be utilized.

### SUMMARY OF THE INVENTION

Liver function can be substituted by metabolically active hepatocytes to reduce toxic buildup in the blood. The inventors have generated hepatocytes from human iPSCs, which may maintain metabolic function in the hollow fiber based bioartificial device of the present invention. Conventional biologic liver support systems utilize human hepatocytes (cadaveric primary hepatocytes or hepatic cancer cells from HepG2 cell line) or xeno-hepatocytes (pig or dog), as cell source. However these cell sources are a significant limitation of these systems, owing to, among other things, the quality and risk of using either pig hepatocytes or human cancer cells. Animal cells confer a risk of zoonotic disease transmission, making human cells more acceptable. However, HepG2-based human tumor cells pose a theoretic risk for transmission of malignancy through leaky membrane and paracrine factors secreted into the circulation. Moreover, besides human cancer cells, human hepatocytes are scarce.

Alternative approaches to liver transplantation include non-biological (hemodialysis and hemoadsorption) as well as biological liver support systems such as ex vivo liver perfusion and hepatocyte systems (human or pig cells loaded devices). For example, the extracorporeal liver assist device (ELAD), HepatAssist liver support system, molecular adsorbent recirculating system (MARS), and the Prometheus device have been in clinical evaluation. To date, non-biological liver support systems have been shown to be ineffective and, while intuitively more promising, no truly effective biologic device has yet to be developed for routine clinical treatment purpose.

In accordance with various embodiments of the present invention, one potential source of human hepatocytes is induced pluripotent stem cells. Metabolically active human hepatocytes derived from a well-characterized iPSC line are believed to offer a tremendous advantage over current BAL cell sources. For instance, a nearly unlimited supply of hepatocytes could be provided by this method. In some embodiments, either autologous or allogeneic iPSC-hepatocytes (iHeps) can be used. In some embodiments, the inventors have developed a BAL module comprised of iPSC-hepatocytes arrayed on the extracapillary space of hollow fibers that allow the flow of blood or plasma through the intracapillary space, thus mimicking the tissue micro-architecture of blood capillaries exchanging gas, nutrients, and metabolites to and from cells. Using the BAL module, the inventors have developed an example of an iPSC differentiation protocol that yields a homogenous population of functional hepatocytes that maintained plasma protein and glucose secretory functions in a hollow fiber bioreactor device.

In some embodiments, disclosed are differentiation protocols that have three phases with various factors applied in each phase that are summarized by the following chart:

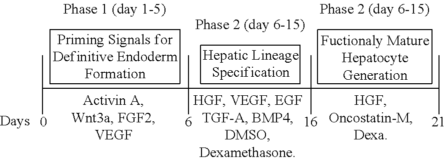

In Phases 1 and 2, the iPSCs are incubated in a two dimensional medium environment. In some embodiments, on day 19 or other suitable days, the iPSCs are harvested for culture in a hollow fiber bioreactor where they are subject to continuous flow of oxygenated media:

The inventors have shown that culturing the cells in a continuous flow hollow fiber bioreactor from day 19-31, in some embodiments, reduces the expression of AFP, the absence of which indicates mature hepatocytes. Thus, the inventors have shown that culturing or maturing of induced hepatocytes in a continuous flow hollow fiber bioreactor may provide superior maturation of hepatocytes. In other embodiments, the inventors have shown that culturing induced pluripotent hepatocytes, or maturing them attached to microcarriers may also yield superior maturation of iPSC hepatocytes.

Below are tables that provide an example of the media formulations for a hepatocyte differentiation protocol:

| 1. | SFD medium | 50 ml | |
|---|---|---|---|
| | IMDM (75%) | 37.5 | ml |
| | Ham's F12 (25%) | 12.5 | ml |
| | 0.5 × N2 Supplement | 250 | μl |
| | 0.5 × B27 without retinoic acid | 500 | μl |
| | Penicillin-Streptomycin | 1000 | μl |

| 2. | Day 1 Medium | 20 ml | | 25 ml | |
|---|---|---|---|---|---|
| | RPMI (90%) | 18 | ml | 22.5 | ml |
| | SFD medium (10%) | 2 | ml | 2.5 | ml |
| | Mouse Wnt 3a (40 ng/ml) | 8 | μl | 10 | μl |
| | Activin A (100 ng/ml) | 4 | μl | 5 | μl |

| 3. | Day 2-3 Medium | 40 ml | | 50 ml | |
|---|---|---|---|---|---|
| | RPMI (100%) | 40 | ml | 50 | ml |
| | BMP4 (0.5 ng/ml) | 20 | μl | 25 | μl |
| | bFGF (10 ng/ml) | 4 | μl | 5 | μl |
| | Activin A (100 ng/ml) | 8 | μl | 10 | μl |
| | VEGF (10 ng/ml) | 2 | μl | 2.5 | μl |

| 4. | Day 4 Medium | 40 ml | | 50 ml | |
|---|---|---|---|---|---|
| | SFD (50%) | 20 | ml | 25 | ml |
| | IMDM (50%) | 20 | ml | 25 | ml |
| | BMP4 (0.5 ng/ml) | 20 | μl | 25 | μl |
| | bFGF (10 ng/ml) | 4 | μl | 5 | μl |
| | Activin A (100 ng/ml) | 8 | μl | 10 | μl |
| | VEGF (10 ng/ml) | 2 | μl | 2.5 | μl |

| 5. | Day 5 Medium | 40 ml | | 50 ml | |
|---|---|---|---|---|---|
| | SFD (75%) | 30 | ml | 37.5 | ml |
| | IMDM (25%) | 10 | ml | 12.5 | ml |
| | BMP4 (0.5 ng/ml) | 20 | μl | 25 | μl |
| | bFGF (10 ng/ml) | 4 | μl | 5 | μl |
| | Activin A (100 ng/ml) | 8 | μl | 10 | μl |
| | VEGF (10 ng/ml) | 2 | μl | 2.5 | μl |

| SFD medium + Ascorbic acid + 1-thioglycerol | 50 ml | |
|---|---|---|
| IMDM (75%) | 37.5 | ml |
| Ham's F12 (25%) | 12.5 | ml |
| 0.5 × N2 Supplement | 250 | μl |
| 0.5 × B27 without retinoic acid | 500 | μl |
| Ascorbic acid | 50 | μl |
| 1-thioglycerol (4.5 × 10⁻⁴M) | 2 | μl |
| Pen-Strep | 1000 | μl |

| 6. | Day 6-11 Medium (6 Days) | 50 ml | |
|---|---|---|---|
| | SFD medium (Ascorbic acid + 1-thioglycerol) | 50 | ml |
| | BMP4 (50 ng/ml) | 25 | μl |
| | bFGF (10 ng/ml) | 5 | μl |
| | VEGF (10 ng/ml) | 2.5 | μl |
| | EGF (10 ng/ml) | 2.5 | μl |
| | TGF α (20 ng/ml) | 5 | μl |
| | HGF (100 ng/ml) | 25 | μl |
| | Dexamethasone (1 × 10⁻⁷M) | 5 | μl |
| | DMSO 0.5% | 250 | μl |

| 7. | Day 12-15 Medium (4 Days) | 50 ml | |
|---|---|---|---|
| | SFD medium (Ascorbic acid + 1-thioglycerol) | 50 | ml |
| | bFGF (10 ng/ml) | 5 | μl |
| | VEGF (10 ng/ml) | 2.5 | μl |
| | EGF (20 ng/ml) | 10 | μl |
| | HGF (100 ng/ml) | 25 | μl |
| | Dexamethasone (2 × 10⁻⁷M) | 10 | μl |
| | DMSO 1% | 500 | μl |

| 8. | Day 16-21 Medium (6 Days) | 50 ml | |
|---|---|---|---|
| | SFD (100%) | 50 | ml |
| | HGF (100 ng/ml) | 25 | μl |
| | Oncostatin (OSM) (20 ng/ml) | 10 | μl |
| | Dexamethasone (2 × 10⁻⁷M) | 10 | μl |
| | Non-essential amino acid (NEAA) 1% | 500 | μl |

The technology may be implemented in a bioartificial liver module by seeding the iPSCs into the extracorporeal space of a hollow fiber bioreactor. In some embodiments, blood or plasma could then be pumped through the intracapillary space of the bioreactor to allow the induced hepatocytes to exchange gas, nutrients, and metabolites to and from the cells.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, NY 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July 6(7):511-9; Queen and Selick, Humanized immunoglobulins, U.S. Pat. No. 5,585,089 (1996 Dec); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Indeed, the present invention is in no way limited to the methods described herein. For purposes of the present invention, the following terms are defined below.

“Subject” as used herein includes all animals, including mammals and other animals, including, but not limited to, companion animals, farm animals and zoo animals. The term “animal” can include any living multi-cellular vertebrate organisms, a category that includes, for example, a mammal, a bird, a simian, a dog, a cat, a horse, a cow, a rodent, and the like. Likewise, the term “mammal” includes both human and non-human mammals.

“Therapeutically effective amount” as used herein refers to the quantity of a specified composition, or active agent in the composition, sufficient to achieve a desired effect in a subject being treated. A therapeutically effective amount may vary depending upon a variety of factors, including but not limited to the physiological condition of the subject (including age, sex, disease type and stage, general physical condition, responsiveness to a given dosage, desired clinical effect) and the route of administration. One skilled in the clinical and pharmacological arts will be able to determine a therapeutically effective amount through routine experimentation.

“Treat,” “treating” and “treatment” as used herein refer to both therapeutic treatment and prophylactic or preventative measures, wherein the object is to prevent or slow down (lessen) the targeted condition, disease or disorder (collectively “ailment”) even if the treatment is ultimately unsuccessful. Those in need of treatment may include those already with the ailment as well as those prone to have the ailment or those in whom the ailment is to be prevented.

“Stem cell” as used herein refers to a cell that can continuously produce unaltered daughters and also has the ability to produce daughter cells that have different, more restricted properties. Stem cells include adult and ES cells.

“Progenitor cell” as used herein includes liver cells, as well as cells that have attributes and characteristics of liver cells, such as the expression of markers associated with liver progenitor cells.

“Packaging material” as used herein refers to one or more physical structures used to house the contents of a kit, such as inventive compositions and the like. The packaging material is constructed by well-known methods, preferably to provide a sterile, contaminant-free environment.

“Package” as used herein refers to a suitable solid matrix or material such as glass, plastic, paper, foil, and the like, capable of holding individual kit components.

Thus, for example, a package can be a cryocontainer used to contain suitable quantities 20 of peritoneal stem cells and/or peritoneal cells described herein. The packaging material generally has an external label which indicates the contents and/or purpose of the kit and/or its components.

As used herein, “FGF” means fibroblast growth factor.

“HES2 and “HES3” as used herein refer to cell lines of human embryonic stem cells.

As used herein, “microcarrier” is a support matrix for attachment and growth of anchorage-dependent cells in suspension systems.
As used herein, “Wnt3 a” means a protein encoded by the WNT3A gene.
As used herein, “VEGF” means vascular endothelia growth factor.
As used herein, “bFGF” means basic fibroblast growth factor.
As used herein, “aFGF” means acidic fibroblast growth factor.
As used herein, “BMP2” means bone morphogenetic protein 2.
As used herein, “BMP4” means bone morphogenetic protein 4.
As used herein, “HGF” means hepatocyte growth factor.
As used herein, “EGF” means epidermal growth factor.
As used herein, “TGF-A” means transforming growth factor alpha.
As used herein, the term “BMP” means bone morphogenetic protein.

As used herein “definitive endoderm (DE)” refers to cells exhibiting the characteristics and morphology including protein or gene expression characteristics of the definitive endoderm.

The present invention is also directed to kits for the induction, propagation and/or isolation of liver progenitor cells. The present invention is also directed toward kits for the transplantation of liver progenitor cells to liver damaged models. Each kit is an assemblage of materials or components. The exact nature of the components configured in each inventive kit depends on its intended purpose. For example, some embodiments are configured for the purpose of inducing and/or propagating hepatocyte precursors. Instructions for use may be included in the kit. “Instructions for use” typically include a tangible expression describing the technique to be employed in using the components of the kit for a desired purpose, such as for induction, propagation, and/or isolation of liver progenitor cells.

Optionally, the kits also contain other useful components, such as those described herein, or buffers (e.g., PBS), growth media, tissue culture plates, multiple well plates, flasks, chamber slides, differentiation media, stem cell media, goat serum, fetal bovine serum, basic fibroblast growth factor, epidermal growth factor, diluents, pharmaceutically acceptable carriers, syringes, catheters, applicators, pipetting or measuring tools, or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s).

Cell Types Utilized for Differentiation into Hepatic Function

In some embodiments, disclosed is a biological liver support system that includes hepatocytes. In general, a biological liver support system is comprised of several distinct components: (a) a cell source: human hepatocytes (cadaveric primary hepatocytes or hepatic cancer cells from HepG2 cell line) or xeno-hepatocytes (pig or dog), (b) a bioreactor to house the cells and (c) a perfusion system for blood or plasma [11, 19, 23]. Hollow fiber capillary bioreactors loaded with hepatocytes of pig origin or HepG2 sub clone C3A cells in the extracapillary space (ECS) for detoxification and plasma protein synthesis purposes have been under clinical investigation [24-27]. In these studies, typically the plasma from a liver failure patient is perfused through the intracapillary space (ICS) of bioreactors in order to be cleared of toxic metabolites by ECS hepatocytes via bidirectional mass transport at the membrane (pore size of 0.1 to 0.2 um) in the semi permeable hollow fiber. In clinical trials, the use of extracorporeal BAL systems have resulted in some improvement in patent's neurological score, blood chemistry, and prothrombin time [11, 17, 26, 27].

The limitations of currently available liver support systems are the quality and risk of using either pig hepatocytes or human cancer cells that are incorporated in the liver support systems [23, 28]. Animal cells confer a risk of zoonotic disease transmission, making human cells more acceptable [29]. Unfortunately, a major bottleneck of such a device is the scarce availability of human hepatocytes. HepG2-based human tumor cells pose a theoretic risk for transmission of malignancy through a leaky membrane and paracrine factors secreted into the circulation [23]. A previous study has shown that HepG2 conditioned media can induce transformation of normal cells [30]. Recent evidence suggests oncogenic microRNAs and proteins are secreted by tumor cells via exosomes [31, 32] and could pose serious health risks.

Therefore, the inventors have determined that metabolically active human hepatocytes derived from a well characterized induced pluripotent stem cell (iPSC) line could offer a tremendous advantage over the current BAL cell sources. In some embodiments, either autologous or allogeneic iPSC hepatocytes (iHeps) can be used. Accordingly, the inventors have developed systems and methods for differentiating and maturing hepatocytes from iPSCs as disclosed herein.

### Overview of Differentiation Protocol

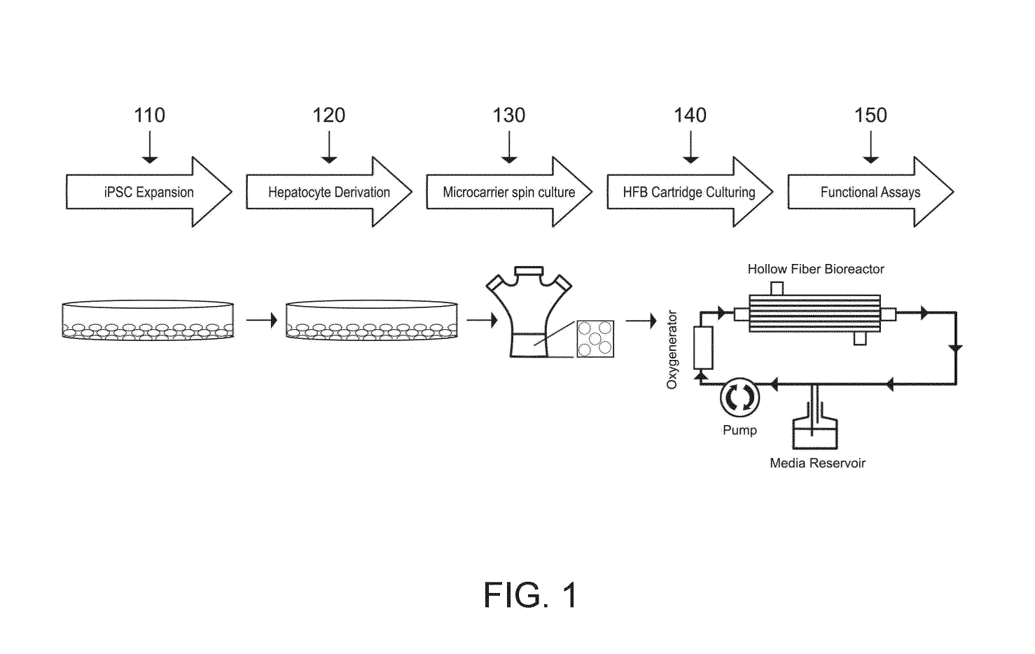

Illustrated in FIG. 1 is a diagram of the different phases of a differentiation protocol utilizing iPSCs. First, the iPSCs are expended 110 to increase the number of iPSCs. Expansion 110 may be performed through any suitable means including by applying any suitable factors. In some embodiments, this will be performed in a 2 dimensional array and not in a bioreactor. In other embodiments, the expansion 110 phase will be performed in a hollow fiber or other bioreactor. The next phase will be hepatocyte derivation 120 where the stem cells will begin to differentiation into hepatocytes. Following derivation, a technician may perform a microcarrier spin culture 130 on the hepatocytes, in order to adhere the hepatocytes to microcarriers. In some embodiments, the microcarriers will then be loaded into a hollow fiber bioreactor (HFB) for further culturing 140. In some embodiments, the hepatocytes may be further cultured on the microcarriers in a different culturing environment. The microcarriers may be added to a continuous flow incubator, a non-hollow fiber continuous flow incubator, or other incubators. In other embodiments, the hepatocytes may be cultured in the bioreactor without being added first to microcarriers. Once the hepatocytes have been cultured in the hollow fiber bioreactor 140, they may be moved towards functional assays 150 to determine their expression of AFP and other proteins or factors that would indicate they are functioning liver cells.

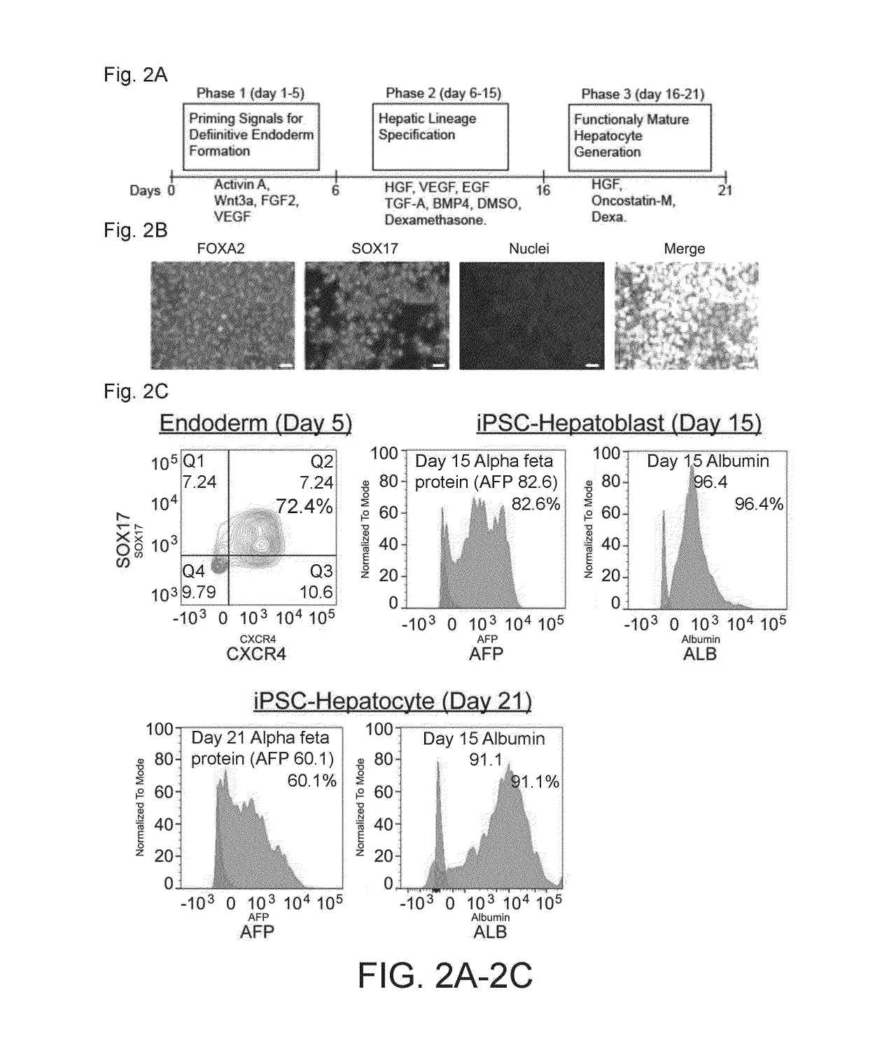

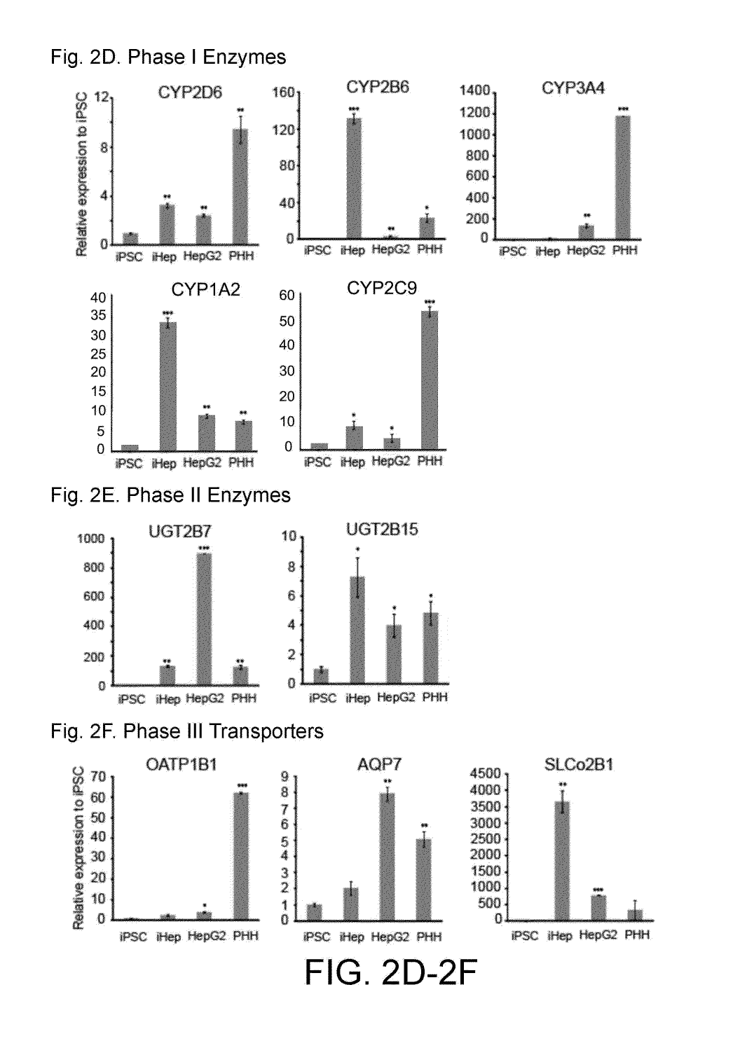

In some embodiments, the differentiation protocol may be roughly broken into 3 phases that can be defined as: (1) phase 1: endoderm induction, (2) phase 2: hepatic specification, and (3) phase 3: hepatic maturation. Following is a description of the specific protocols and factors that may be utilized that roughly fall within each of these three phases. In other embodiments, various phases may overlap, be different lengths, or have factors that span multiple phases, or be broken into additional phases. These three phases are illustrated in the timeline represented in FIG. 2A. This example of a timeline runs from day 0 through day 21, and is only intended as an exemplary example. Other timelines of various lengths and with different factors may be utilized.

### Differentiation Protocol Phase 1: Endoderm Induction

FIG. 2A illustrates Phase 1 of the differentiation protocol that is responsible for differentiation of the stem cells toward definitive endoderm (DE). In some embodiments, this is induced using high concentrations of activin A. Various other factors may be applied during the initiation phase to induce endoderm formation, including, Wnt3a, FGF2, VEGF, BMP-4 and other factors. In some embodiments, Phase 1 may last 3, 4, 5, 6, or 7 days. In other embodiments, other factors may be utilized including bFGF, Wnt3a agonist CHIR99021 (CHIR), Glutamax-I, aFGF, BMP2, BMP4, and FCS in combination with the other factors disclosed herein including activin A.

### Differentiation Protocol Phase 2: Hepatic Specification

FIG. 2A also illustrates Phase 2 of the differentiation protocol which, in some embodiments, results in the specification of the hepatic lineage from the endoderm germ layer tissue formed in Phase 1. In some embodiments, various factors may be utilized including HGF, VEGF, EGF, TGF-A, BMP4, DMSO, Dexamethasone, and other factors in various combinations. In some embodiments, BSA, Ascorib acid, Glutamax-I, D-Galactose/D-sorbitol, Hydrocortisone, Insulin, Transferrin, BMP2, aFGF, and bFGF may be utilized. Phase 2 may last 5, 6, 7, 8, 9 or other suitable number of days. In some embodiments phase 2 will last from day 6-15.

### Differentiation Protocol Phase 3: Hepatic Maturation

FIG. 2A also illustrates Phase 3 of the differentiation protocol that results in functionally mature hepatocytes. In some embodiments, various factors may be utilized in various combinations to mature the hepatocytes, including HGF, Oncostatin-M, Dexamethasone, and other factors. In some embodiments, other factors may be utilized including Ascorbic acid, Glutamax-I, Dexamethasone, D-Glactose/D-sorbitol, Hydrocortisone, Insulin, Transferrin, bFGF, and EGF. Phase 3 may be 4, 5, 6, 7, 8, 20, 30 days, or other suitable numbers of days. In some embodiments, Phase 3 may last until AFP expression decreases to an acceptable level (e.g. decreases 50 fold, 90 fold, 100 fold, 40 fold, etc.) that indicates mature hepatocytes. In some embodiments, Phase 3 may be from day 16-day 21.

### Bioreactor Incubation

In some embodiments, certain portions of the differentiation protocol or the entire protocol may be performed in a bioreactor. In these embodiments, the cells of interest are loaded into the bioreactor and media is perfused through the capillaries. In some embodiments, this may be a hollow fiber bioreactor, a continuous flow hollow fiber bioreactor, or other suitable incubators. In some embodiments, a polysulfone hollow fiber Bioreactor cartridge (e.g., as available from Alpha Plan, Germany) with 70-6000 cm² inner surface area and 0.21 μm pore size may be used for iHep culture. In some embodiments, the bioreactor may contain microporous polyethersufone capillary membranes (e.g., as available from mPES, Membrana, Wuppertal, Germany) or multi-laminate hollow fiber capillaries (e.g., as available from MHF, Mitsubishi, Tokyo, Japan) to enable gas exchange with the cells. In some embodiments, the hollow fibers are arranged in longitudinal fashion with a pack density of 36%-40%, 20%, 50%, or other suitable percentages without waviness inside durable polycarbonate casing of the cartridge. In some embodiments, the fibers may be wavy.

The HFB cartridge may be connected to a reservoir bottle or other container containing growth media, a membrane oxygenator and a pump with peroxide cured silicon tubing or other suitable tubing for incubation. The media from reservoir pumped into the HFB cartridge's vestibule is divided across the hollow fiber mouth for even flow into the ICS of fibers. Before entering into the cartridge, the media is oxygenated (95% O₂ and 5% CO₂) by an oxygenator module. This closed circuit HFB system was placed in a cell culture incubator at 37° C. In some embodiments, the flow rate of the media in the bioreactor may be 1 ml/minute, 2 ml/minute, 22-30 ml/minute, 40 ml/minute, and may altered depending on the stage of the incubation.

Additionally, in some embodiments, the hepatocytes and/or progenitor cells may be adhered to microcarriers prior to introduction into the extracapillary space of the bioreactor. Microcarriers are a support matrix for attachment and growth of anchorage-dependent cells in suspension systems. They provide high surface area to volume ratios for facilitating a scale-up process of generating hepatocytes. In other embodiments, the hepatocyte cells may be incubated in the bioreactor without microcarriers, for example as free cells or cell clumps (organoids) in the extracapillary space.

In some embodiments, the microcarriers may be collagen coated. In other embodiments, they may be coated with other extra-cellular matrix proteins and small molecules. In some embodiments, the microcarriers may be porous gelatin microcarriers with a diameter of 9-100 um. In some embodiments, gelatin and cytodexill microcarriers may be utilized. In other embodiments, micro carriers may be porous or nonporous, comprised of gelatin, glass, collagen, or cellulose, and have dimensions from 170 μm-6,000 μm. In some embodiments, a pore size of less than 1 μm, 1 μm, 3 μm, 4μm, or 5 μm may be utilized. In some embodiments, the immature hepatocytes may be harvested on day 15, 16, 17, 18, 19, 20, 21, or 22 or other suitable day for culture in a hollow fiber bioreactor. In other embodiments, the hepatocytes may be cultured from day 10 on in a hollow fiber bioreactor. In other embodiments, the iPSCs may be cultured from day 1 onward in the bioreactor.

### Bioartificial Liver Device

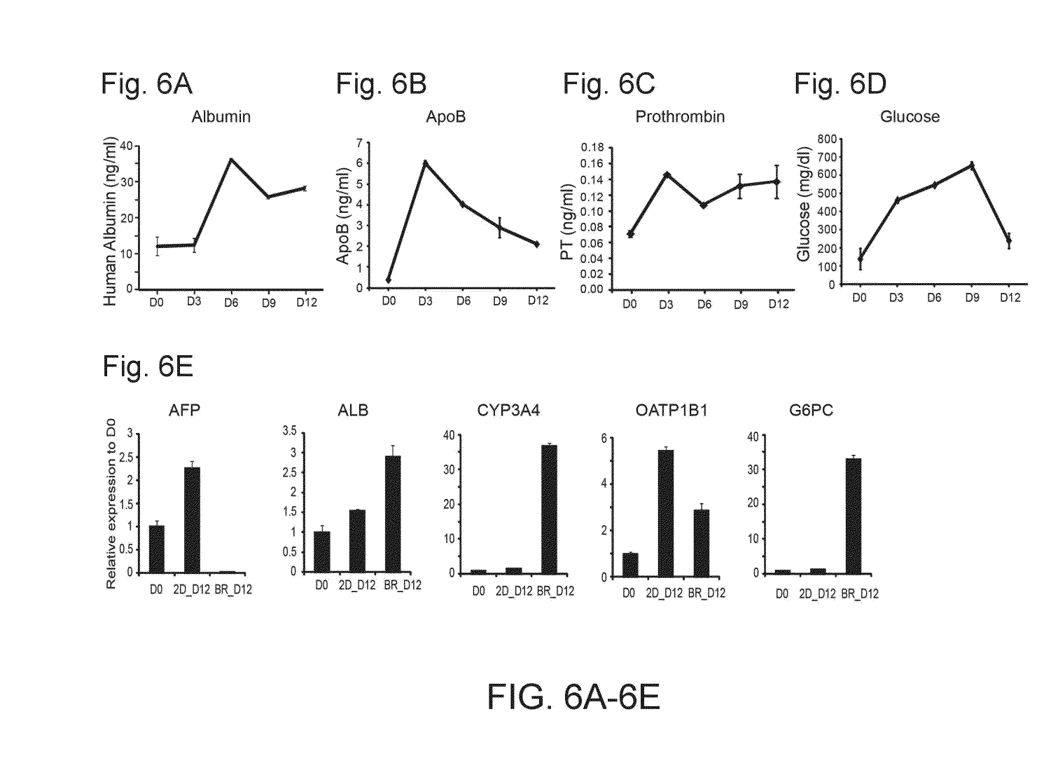

In general, hepatic cells derived from pluripotent stem cells using current differentiation protocols are immature as demonstrated by presence of alpha fetoprotein (FIG. 2C). For example, in adult liver, AFP expression is below detectable. The inventors observed that the iHeps cultured in the hollow fiber cartridge with continuous flow showed approximately a two order of magnitude reduction in AFP expression with concomitant increase in mature liver marker (FIG. 6). This finding has great practical value as mature hepatocytes are critical for assessing the toxicity and pharmacokinetic properties of drug compounds during preclinical development phase. Moreover, iHeps matured under continuous flow conditions can be a valuable cell source for cell therapy application towards inherited liver metabolic disorders. Accordingly, in some embodiments, these differentiation protocols disclosed herein may be utilized to derive i-Heps for a bioartificial liver device.

In some embodiments, a bioartificial liver device may include hollow fibers. A hollow fiber capillary design is utilized for its high surface area and resemblance to natural blood capillary liver microarchitecture for nutrient and gas exchange. This design has been used in other liver support devices such as extracorporeal liver assist device (ELAD), and HepatAssist liver support system [11, 26]. Additional design features such as cartridge having layers of polysulfone membrane sheets where alternate layers filled with cells or blood/plasma flow can be considered. Another design consideration is improving the throughput. In some embodiments, a single cartridge closed circuit system may be utilized. For testing multiple growth conditions, animal experiments, and human clinical studies, a multi-cartridge system can be useful. An integrated multi-cartridge system can allow for parallel production, better quality control and uniformity among bioartificial liver devices.

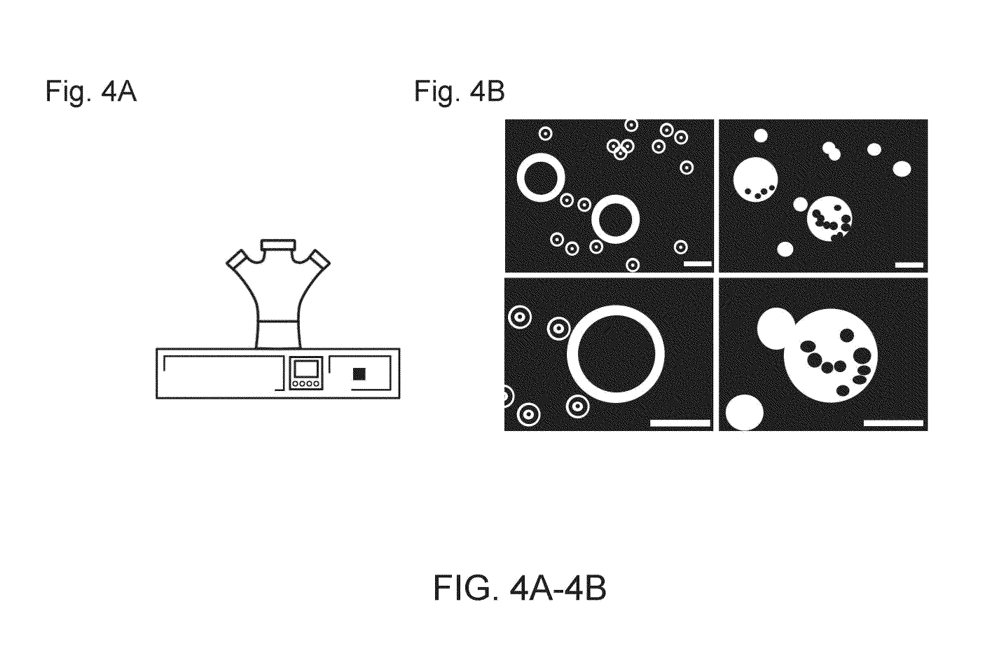

In some embodiments, the inventors utilize dextran microcarrier beads for growing iHeps in suspension culture (FIG. 4). An advantage of using microcarrier suspension, in addition to the potential for large scale culturing, is that iHep-beads can be easily transferred between culture modules without having the stress of an enzymatic dissociation process such as trypsinization. In some embodiments, as disclosed herein, a variety of different microcarrier beads may be utilized. The iHep microcarrier culture approach can provide additional benefit such as minimizing clogging of pores in the hollow fiber by cells. If the cells are directly attaching and expanding on the surface of hollow fiber, there is a high possibility of pore obstruction which can prevent the exchange of gas, metabolites and nutrients in and out of the fiber capillaries.

The BAL devices disclosed herein may be further developed and validated for rescuing disease specific decompensated liver functions. In acute liver failure due to acetaminophen (APAP; paracetamol) poisoning, the liver cannot detoxify ammonia and secrete blood coagulation proteins such as prothrombin resulting in increased intracranial pressure and bleeding [64, 65]. For this purpose, APAP metabolism and lethal concentration, and ammonia detoxification by iHeps needed to be assessed. Moreover, unbiased analysis of metabolites and proteins that are secreted by iHeps can be conducted using a mass spectrometer. The secreted liver proteins and metabolites can be used as biomarkers for defining the therapeutic use window of an iHepBAL in testing the efficacy in an animal and human.

For clinical applications, the iHepBAL device has to reconstitute the function of the damaged liver and improve the health condition of the patient. In vivo preclinical studies in the rat and pig model systems of liver failure will provide insights into safety and efficacy of an iHepBAL device. For preclinical testing, the following acute liver failure animal models, anhepatic rat and acetaminophen toxicity in pig, can be used [66, 67]. Based on plasma proteins, albumin and apoB, secretion data (FIG. 6), the iHeps can be cultured for 2 days in the cartridge after loading and subsequently be used for liver support purpose for in vivo testing. In some embodiments, a 70 cm² minicartridge may be utilized. For testing the efficacy of an iHepBAL in a large animal setting, a larger HFB cartridge (6000 cm²) with a loading density of over 1×10⁹ cells may be utilized. Scaling up of iHep production is a key component, which can be accomplished by differentiating and expanding cells on microcarriers in a suspension culture.

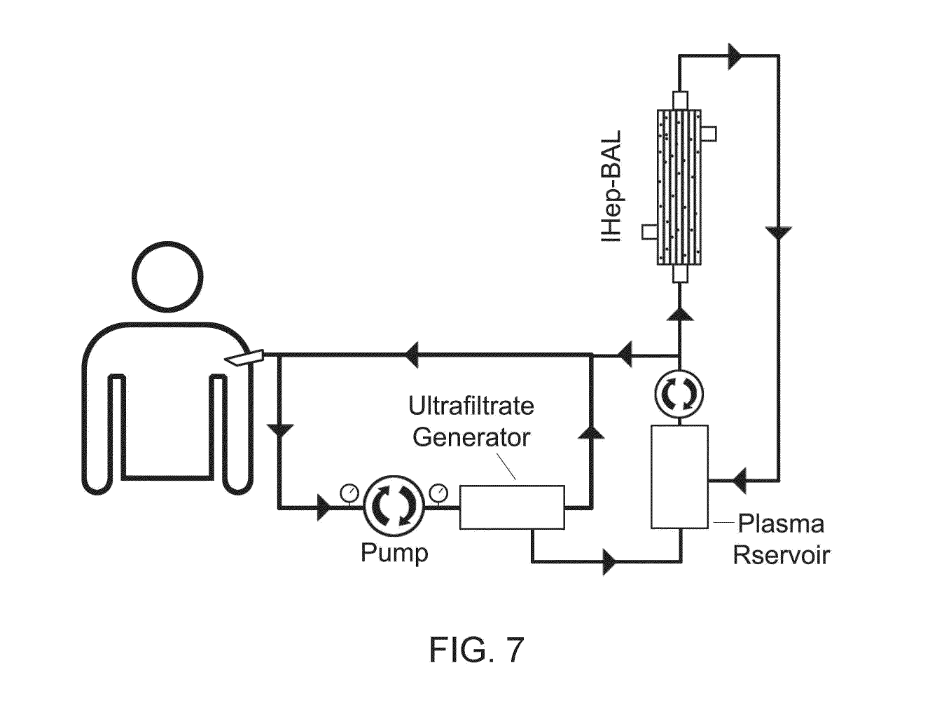

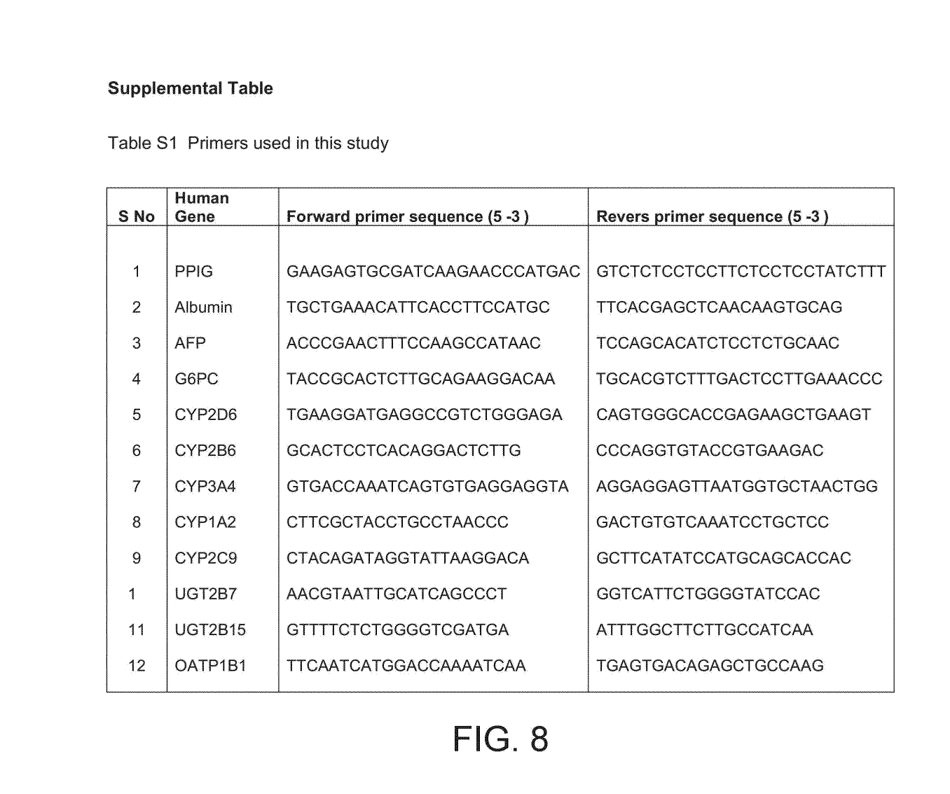

FIG. 7 shows a concept outline of iHep based extracorporeal closed circuit BAL system for supporting liver failure patient, where the patient's plasma will be circulated through the iHep loaded cartridge for detoxification and plasma protein reconstitution. In some embodiments, plasma separated from a patient's blood will be passed through a hollow fiber cartridge containing iHeps that will remove toxic metabolites. The plasma will be separated from the blood using an ultrafiltrate generator. In some embodiments, the plasma source may include its own pump for pumping the plasma through the iHep based bioartificial liver device.

In some embodiments, the plasma lines may be divided or separated into multiple parallel lines to be circulated through multiple iHep-BAL devices to improve throughput. In some embodiments, different rates of filtration may be utilized for different amounts of liver support needed. In other embodiments, whole blood may be passed through a bioartificial liver device rather than first separating the plasma from the blood. In other embodiments, hollow fibers may not be used and instead the plasma may be in direct contact with the iHeps. For example, the Academisch Medisch Centrum Bioartificial Liver developed by Chamuleau's group utilizes this configuration. Various other configurations of a bioartificial liver device may be utilized that include iHeps differentiated and cultured accordingly to the methods and in accordance with the devices disclosed herein.

### EXAMPLES

The following examples are provided to better illustrate the claimed invention and are not intended to be interpreted as limiting the scope of the invention. To the extent that specific materials or steps are mentioned, it is merely for purposes of illustration and is not intended to limit the invention. One skilled in the art may develop equivalent means or reactants without the exercise of inventive capacity and without departing from the scope of the invention.

### Example 1

### General Methods

Generally, an example of a multi-step differentiation protocol for generating of hepatocytes is shown in FIG. 1. First, the iPSCs are expended 110 in a 2 or 3 dimensional array. Next, the cells are cultured to achieve hepatocyte derivation 120. Following derivation, a technician may perform a microcarrier spin culture 130 on the hepatocytes, in order to fix the hepatocytes on microcarriers. In some embodiments, then, the microcarriers will be loaded into a hollow fiber bioreactor (HFB) for further culturing 140 and maturation.

### Example 2

### Cells and Reagents

In this example experiment, the human iPSC line 83iCTRL was obtained from Cedars Sinai Medical Center iPSC core facility [33]. The iPSC 83iCTRL line was established by reprogramming normal human fibroblast using non-integrating expression vector carrying OCT4, SOX2, KLF4, and L-MYC genes. Human embryonic stem cell (hESC) line, WA09 (H9), was obtained from WiCell Research Institute, USA. The iPSCs and hESCs were cultured using serum free chemically defined media, mTeSR1, (STEMCELL Technologies, Canada) with daily media change regimen at 37° C. incubator with 5% CO₂. The human liver cancer cell line HepG2 and Huh7.5.1 was maintained on complete Dulbecco's modified Eagle's medium (DMEM) (Fisher Scientific). Complete DMEM was supplemented with 10% fetal bovine serum (FBS), 10 mM Hepes, 10 mM nonessential amino acids, penicillin (100 units/ml), streptomycin (100 mg/ml), and 2 mM Lglutamine (Life Technologies).

### Example 3

### In Vitro Differentiation of Human iPSCs into Hepatic Lineage Cells

For in vitro differentiation, the iPSCs were single cell plated in a 6 well plate, cultured at 37° C. in 5% CO₂ and subjected to a 3 week hepatic differentiation protocol. The differentiation steps consisted of three phases, including endoderm induction (day 1-5), hepatic specification (day 6-15) and hepatic maturation (day 16-21). The cytokines were purchased from Peprotech Inc., (Rocky Hill, N.J.) unless otherwise mentioned. The cells were differentiated to endoderm for 5 days using IMDM/F12 or RPMI media (Life Technologies) supplemented with Wnt 3A (40 ng/ml, R and D Systems) and Activin A (100 ng/ml) for the first one day and then treated with Activin A, VEGF (10 ng/ml) and bFGF (10 ng/ml) for an additional 4 days. From day 6 onwards, the media was changed to IMDM/F12 supplemented with BMP4 (50 ng/ml), VEGF (10 ng/ml), EGF (10 ng/ml), TGFα (20 ng/ml), HGF (100 ng/ml), dexamethasone (1×107 M; SigmaAldrich, St. Louis, Mo.) DMSO (1%, SigmaAldrich). From day 12 onwards, BMP4 and TGFα were removed from the cocktail. For hepatocyte maturation, HGF, dexamethasone and oncostatin M (20 ng/ml) were included in the media from day 16 onwards. At day 19 post differentiation, the iPSC hepatocytes or iHeps were harvested for bioreactor culture. To compare the hepatic maturation status between bioreactor cultured iHeps and two dimensional monolayer cultured iHeps, one plate of the cells was continuously differentiated until day 31 in parallel to bioreactor culture. At specific time points, markers for endoderm and hepatic lineage cells were assessed by immunocytochemistry (ICC), flow cytometry and reverse transcription quantitative PCR (RTqPCR).

### Example 4

### Closed Circuit Hollow Fiber Bioreactor (HFB) System for Culturing iHeps

Preparation of HFB system: The closed circuit bioreactor system comprised a media reservoir, pump, oxygenator, HFB cartridge containing cells, and flow path (tubing and adaptors). A polysulfone hollow fiber Bioreactor cartridge (Alpha Plan, Germany) with 70 cm² inner surface area and 0.21 μm pore size was used for iHep culture. The hollow fibers are arranged in longitudinal fashion with a pack density of 36%-40% without waviness inside durable polycarbonate casing of the cartridge. The HFB cartridge was connected with a reservoir bottle (FiberCell Systems Inc., USA) containing growth media, a membrane oxygenator (Radnoti, LLC., USA) and a pump (Masterflex L/S Digital Drive pump; ColeParmer, USA) by peroxide cured silicon tubing (ColeParmer, USA). The media from reservoir pumped into the HFB cartridge's vestibule is divided across the hollow fiber mouth for even flow into the ICS of the fibers. Before entering into the cartridge, the media is oxygenated (95% O₂ and 5% CO₂) by an oxygenator module. This closed circuit HFB system was placed in a cell culture incubator at 37° C.

Preculturing iHeps on microcarrier beads in spinner flask: A total of 9 to 10 million iHeps (day 19 post differentiation) were harvested from the monolayer culture plate, and precultured with laminin coated Cytodex microcarrier beads (Sigma Aldrich) in 150 ml HepatoZymeSFM (Life Technologies) media supplemented with ITS (InsulinTransferrin Selenium, Life Technologies) and EGF (25 ng/ml) in a spinner culture bottle. The spinner bottle culture was performed in a CO2 incubator at 37o C with the stir speed of 40-70 RPM.

Loading iHeps into HFB system: Prior to cell loading, the bioreactor system was primed by perfusion with 500 ml of PBS for 24 hours, then by 500 ml of HepatoZyme-SFM media for 48 hours. For culturing cells, 250 ml of HepatoZyme-SFM media supplemented with 2.5 ml of ITS, EGF (25 ng/ml) was perfused. Before cell loading, the extracapillary space of the bioreactor was coated with the mouse laminin (Mouse Laminin I, Trevigen) according to the manufacture's protocol. After 24 hours of preculturing in a spinner bottle, the cells on the microcarrier beads were harvested by centrifugation, resuspended in 3 ml of HepatoZyme-SFM medium, and loaded into the extracapillary space of the HFB cartridge with a syringe. The media flow rate was set at 2 ml/min. At indicated time points (day 0, 3, 6, 9, and 12) post cell loading, 25 ml of the culture medium was collected from the bioreactor media reservoir bottle, and replaced with same volume of fresh made HepatoZyme-SFM medium containing ITS and EGF. The collected medium was stored at 20° C. for future assays. At day 12 post loading, the cells in the ECS of the bioreactor were also harvested for RNA isolation.

### Example 5

### Reverse Transcription Quantitative PCR Analysis

Total RNA was extracted from iHeps cultured in plates or bioreactor using RNeasy Mini Kit (QIAGEN). Human primary hepatocyte RNA from fetal liver was included as a positive control (provided by Samuel W French, UCLA). RNA samples were reverse transcribed using SuperScript III Reverse Transcriptase kit (Life Technologies) with random primers as described by the manufactures. Expression of human hepatic genes was quantified using Platinum SYBR Green qPCR SuperMixUDG with ROX Kit (Invitrogen) by the ViiA7 realtime PCR system (Applied Biosystems). Approximately 100 ng of cDNA was used for each qPCR reaction. The reaction for each marker gene expression was triplicated with the housekeeping human PPIG (cyclophilin G) gene as an internal reference control. The sequences of the primer pairs used for qPCR are given in supplementary Table S1. All cDNAs were amplified under the following conditions: 50° C. for 2 min; 95° C. for 2 min followed by 40 cycles of 95° C. for 15 sec and 60° C. for 1 min.

### Example 6

### Human Albumin, Apolipoprotein B, Prothrombin, Glycogen and Glucose Assays

The iHep culture media samples from both bioreactor and culture plate were collected. The amount of human albumin and apolipoprotein B produced by the iHeps was measured in triplicate by the Human Albumin ELISA kit (Bethyl Laboratories, Inc., Montgomery, Tex.) and the Human Apolipoprotein B ELISA kit (MABTECH, Inc) respectively. Prothrombin was measured by ELISA as per manufacturer's protocol (Molecular Innovations, Inc). Intracellular glycogen was detected by Periodic Acid Schiff (PAS) assay as per manufacturer's protocol (SigmaAldrich). Glucose was measured by a hexokinase enzyme based colorimetric assay.

### Example 7

### Flow Cytometry

The differentiating cells were collected at indicated time points, permeabilized using the Cytofix/Cytoperm kit (BD Biosciences) and analyzed using antibodies against SOX17 and CXCR4 (R & D Systems), AFP (Dako, USA) and Albumin (Bethyl Laboratories, Inc) and FOXA2 (Novus Biologicals, Littleton, Colo.). Data was acquired on a BD Fortessa (BD Biosciences) flow cytometer using FACS diva software and analyzed using FlowJo software (TreeStar Inc.).

### Example 8

### Immunofluorescence Assay

Cells were fixed with methanol at day 4 post differentiation. Following three PBS washes, the cells were blocked (10% fetal bovine serum, 3% BSA, 0.1% Tritonx 100 in PBS) and incubated with human SOX17 mouse monoclonal primary antibody (BD Biosciences) and FOXA2 rabbit monoclonal primary antibody (Novus Biologicals, Littleton, Colo.) at a dilution of 1:200 for 5 hours to overnight at 4° C. The goat anti-mouse polyclonal antibody (Alexa fluor 594) or goat anti-rabbit polyclonal antibody (Alexa fluor 488) was added as a secondary antibody (Life Technologies, USA) at 1:1000 dilutions and incubated for 1 hour at room temperature. Between antibody changes the cells were washed thrice with PBS. The nuclei were stained with Hoechst dye (Life Technologies, USA).

### Example 9

### Development of a iPSC-Based Bioartifical Liver

For the development of an iPSC based bioartificial liver, the different components of the device including metabolically active iPSC hepatocytes and the closed circuit hollow fiber bioreactor system need to be integrated optimally. FIG. 1 depicts various steps involved in the development of an iHepBAL device. The inventors utilized a human fibroblast derived iPSC line for generating functional hepatocytes (FIG. 2).

### Example 10

### Differentiation of Human iPS Cells into Hepatocyte-Like Cells

The inventors adapted a three stage differentiation program to differentiate iPSCs to mature hepatic lineage cells (FIG. 2A). This process involves the first stage of definitive endoderm formation, a second stage of hepatic lineage specification and the final third stage of hepatocyte maturation using various cocktails of cytokines and factors [34-37]. The endoderm formation was driven using Activin A, Wnt3a, and FGF2. Activin A activates the activin/nodal signaling pathway critical for definitive endoderm formation (FIG. 2). On days 6 to 15 the cells were grown in the presence of BMP4, HGF, VEGF, EGF, TGFα, FGF2, dexamethasone and DMSO. The final stage of maturation was attained using dexamethasone, HGF and Oncostatin M.

To accurately measure the differentiation efficiency at single cell level, the inventors employed flow cytometry to quantify cells that express endoderm and liver specific markers. The cells were analyzed on day 45 for endoderm specific markers SOX17 and FOXA2 by immunocytochemistry (ICC) (FIG. 2B), as well as by flow cytometry (SOX17 and CXCR4) (FIG. 2C). SOX17 is a transcription factor specific for endoderm cells and CXCR4 is a chemokine receptor involved in cell migration during the gastrulation phase of embryonic development [3840]. By day 15, the inventors observed high levels of expression of alpha fetoprotein (AFP), a marker for immature hepatic cells (FIG. 2C). Liver specific marker albumin was expressed by over 90% of the cells after day 15 suggesting homogenous and efficient differentiation. By day 21 as the hepatic cells proceed to more mature phenotype, we observed reduced expression of AFP. Below we describe the functional evaluation of differentiated iPSC-hepatocytes.

### Example 11

### Functional Analysis of iPSC-Hepatocytes

Generating metabolically active functional hepatocytes from iPSCs is critical for the successful development of an iHep based bioartificial liver device that can provide metabolic and detoxification functions. Therefore, the inventors assessed the activities of cytochrome p450 (CYP), and carbohydrate metabolic pathways in the iHeps. The liver CYP system is crucial for degradation and clearance of endogenous metabolites, hormones and xenobiotics [41-47]. CYP genes are classified as phase I and Phase II enzymes and Phase III transporters. Phase I enzymes function in oxidative reduction reactions and Phase II enzymes act in modifying metabolites by acetylation, sulfation, glucuronidation and glutathione conjugation. Phase III transporters are involved in drug clearance. CYP components have been investigated extensively for their role in metabolism and excretion of pharmaceutical compounds [41-45, 48, 49].

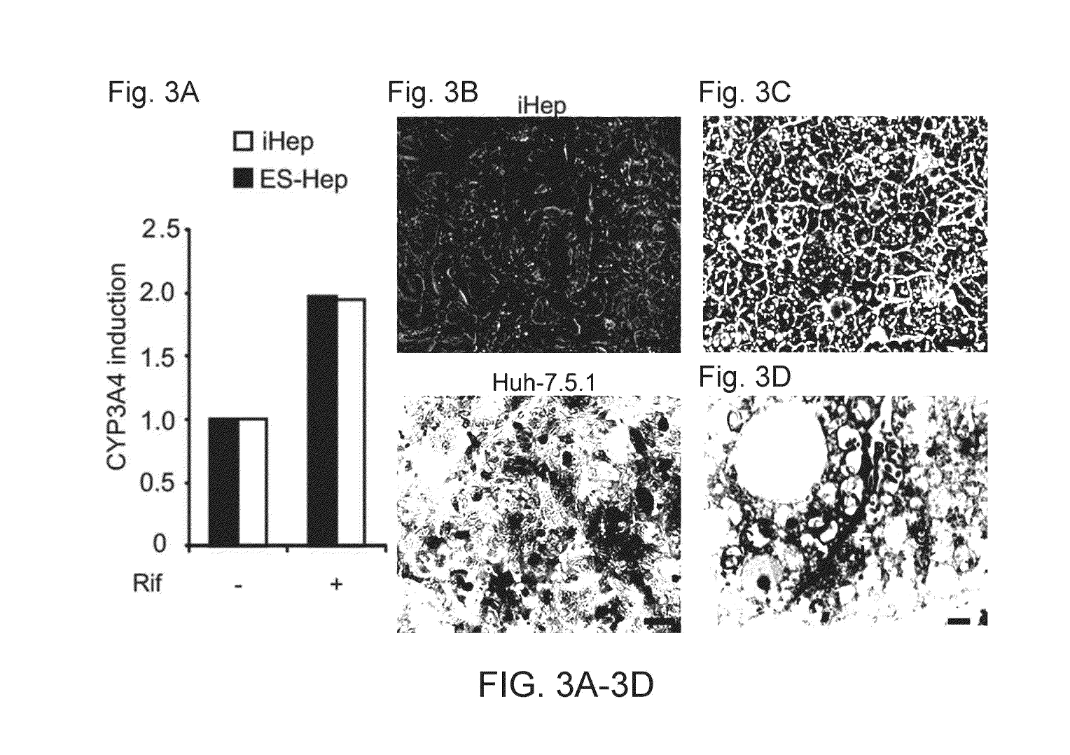

The inventors observed that the basal level expression of many CYP genes, including CYP2D6, CYP2B6, CYP1A2, UGT2B7, UGT2B 15 and SLCo2B 1, were significantly upregulated in iPSC hepatocytes compared to that of undifferentiated iPSCs (FIG. 2D, 2E and 2F). The basal level of CYP3A4 expression was increased in iHep, however was not significant. The inventors also observed that CYP genes were differentially expressed in iHeps, fetal hepatocytes and HepG2. CYP genes are induced upon exposure of hepatocytes to various xenobiotics and drugs. The inventors used the drug rifampicin that is metabolized by the liver to study the CYP activity. The iPSC hepatocytes (20 days post differentiation) were treated with rifampicin (10 μM) for 48 hours and the CYP3A gene expression was quantified. Though iHep had low basal level of CYP3A4, it was induced upon treatment with the rifampicin (FIG. 3A) indicating the metabolic maturity of iHeps.

Glycogen, a branched polysaccharide, is stored in liver cells. To test the capacity of the glycogen storage by the iHeps the inventors performed a Periodic Acid Schiff (PAS) assay. The differentiated iPSC hepatocytes (day 20 post-differentiation) were fixed and stained for glycogen. The intensity of glycogen staining was analyzed. Hepatocytes derived from 83iCTR show glycogen staining, along with the positive control Huh7.5.1 hepatoma cell line (FIG. 3B). The differentiated iPSC hepatocytes exhibited polygonal morphology with tight junctions in monolayer culture condition (FIG. 3C). Ultrastructural analysis demonstrated formation of putative bile canaliculi by adjacent hepatic cells (FIG. 3D). Taken together, the inventors' results showed that the iPSC differentiated hepatocytes exhibit hepatic phenotype and are metabolically active. Next, the inventors focused on utilizing the iHeps for the development of bioartificial liver.

### Example 12

### Design, Process Development and In Vitro Functional Study of Bioartificial Liver

The hollow fiber bioreactor cartridge loaded with functional hepatocytes would serve as a bioartificial liver module. Artificial devices used for compensating the function of failing organs should be capable of replicating essential physiologic functions; thus, the design of the BAL is critical for facilitating the cells to execute respective functions. In liver, plates of hepatocytes are arranged along the sinusoidal capillary spaces where the nutrients, oxygen, metabolites, xenobiotics, hormones, and toxins are exchanged for anabolic and catabolic purposes. Hollow fiber capillary arrayed with iHeps on the outer surface can provide intracapillary flow paths for blood or serum towards metabolic detoxification. The metabolites and waste products from the blood can diffuse through the hollow fiber micropores (size 210 nm) and can be metabolized by cells in the array. The resulting metabolites will be removed by continuous media flow in the hollow fiber bioreactors. With this design, the inventors developed an iHepBAL device.

### Example 13

### Large Scale Culturing of iPSC-Hepatocytes on Microcarriers using Spinner Flask Bioreactor

Large scale production of iPSCs and iPSC hepatocytes is a key step in the development of iHep based BAL. For clinical application, an estimated 5 to 20 billion hepatocytes are required for the treatment of patient with decompensated liver disease. To achieve this, microcarriers are useful for cultivating anchorage dependent cells in suspension which allow large scale and high yield production of iHeps. For a proof of concept study, the inventors used dextran microcarrier beads (6087 Lm in size) for culturing iHeps. The microcarriers were coated with laminin and 10 million iHeps (19 day post-differentiation) were added to the microcarrier in a 250 ml spinner flask (FIG. 4A). Cells were cultured in hepatocyte differentiation media for 24 hours as a suspension culture in a CO₂ incubator at 37° C. Culture samples were collected for microscopic examination. We observed that the iPSC hepatocytes were attached to the surface of microcarrier spheres (FIG. 4B). Trypan blue dye exclusion test indicated that the attached cells were viable. Subsequently, the iHep beads were transferred to continuous flow hollow fiber bioreactor system.

### Example 14

### Hollow Fiber Bioreactor System for Culturing and Functional Assessment of iHep-BAL

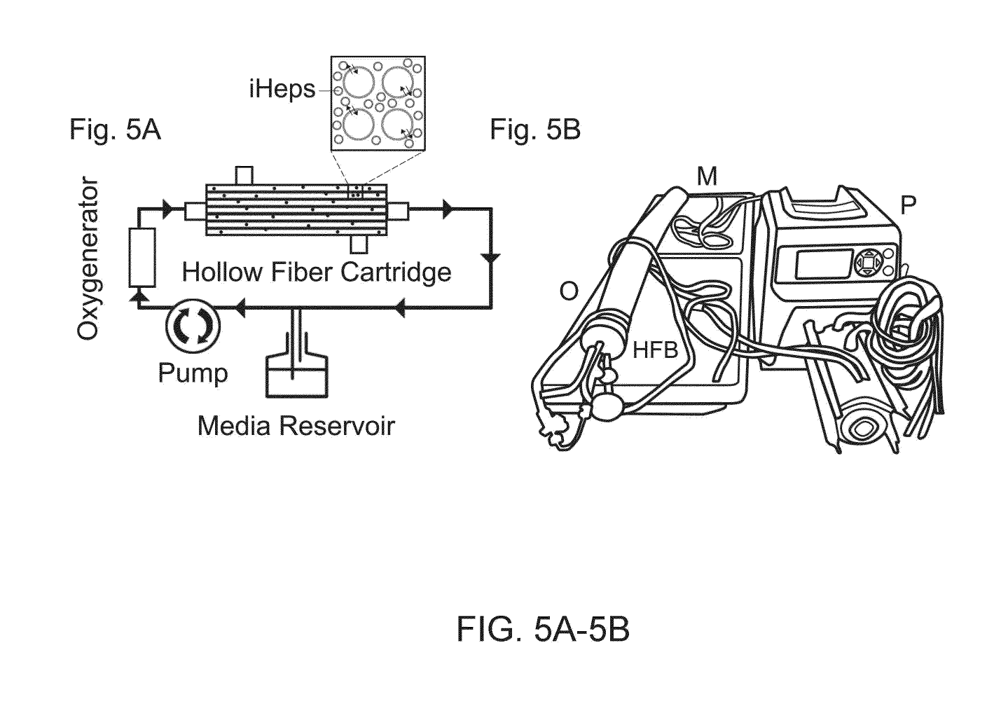

In order to maintain physiologic functions, the iPSC hepatocytes loaded in the bioartificial liver device have to survive in the extracapillary space microenvironment over a period of time and exchange gas, nutrients, proteins and metabolites. These in vitro parameters of cell viability and functions can be used for assessing the quality and functionality of iHepBAL. The iHeps (1×107 cells) attached to the microcarrier beads were loaded into the extracapillary space of polysulfone hollow fiber bioreactor cartridge (70 cm²) using a syringe. We utilized single cartridge closed circuit set up with a perfusion media volume of 250-500 ml (FIGS. 5). The oxygenated culture media was perfused continuously through the intracapillary space at the rate of 2 ml/ minute for a total of 12 days. Media samples were collected from ICS flow through at different time points (days 3, 6, 9 and 12) for functional assays.

### Example 15

### Secretion of Plasma Proteins by iHeps

Liver specific proteins and metabolites produced by the iHeps were secreted into ICS through 0.21 μm pores on the hollow fibers. This design is similar to in vivo natural liver architecture where the hepatocytes secrete plasma proteins into liver sinusoidal spaces. For functional verification, human plasma proteins albumin, apoB and prothrombin secreted by iHeps into the ICS media were quantified (FIGS. 6A, 6B and 6C). We observed a significant increase in human albumin secretion after day 3 of iHep culturing in the cartridge. ApoB secretion was detected from day 3 onwards. Glucose concentration in the flow through was significantly higher compared to that of day 0 input media suggesting glycolytic activity of iHeps (FIG. 6D). Liver enzyme ALT was below detectable levels at all time points indicating better cell survival (data not shown).

### Example 16

### Improved Maturation of iHeps by Continuous Media Flow

In vivo, the generated metabolites and toxic waste products by hepatocytes are continuously removed by the blood flow resulting in maintenance of liver homeostasis. The closed circuit flow system can mimic the in vivo physiological condition compared to that of cells cultured in a two dimensional static condition. Thus, the inventors further characterized the iHeps cultured in the hollow fiber bioreactors. At the end of the experiment on day 12, viable cells (over 80%) were recovered from the bioreactors and used for gene expression analysis. Culturing iPSC hepatocytes in a continuous flow microenvironment improved the maturation state as evidenced by 83 fold reduction of AFP and significant increase in CYP3A4, and Glucose6phosphatase (G6PC) gene expression compared to Day 0 iHeps (FIG. 6E). ALB and OATP1B1 expression was not significantly altered in continuous flow condition. The hepatic markers CYP3A4 and G6PC were also upregulated in continuous flow cultured iHeps compared to that of parallel static 2D plate cultured iHeps. In conclusion, the inventors' results indicated that the iPSC hepatocytes cultured in the HFB maintained the plasma protein secretory function with an improved hepatic maturation signature.

### Example 17

### Proof-of-Concept Preclinical In-Vitro Study on Development of a Prototype iPSC-Hepatocyte-Based Bioartifical Liver Module

The biological factors and chemicals required for deriving functional hepatocytes from pluripotent stem cells have been studied extensively [34-37, 50-61]. Using the established three phase differentiation protocol, the inventors have generated over 90% homogenous population of hepatic lineage cells (FIG. 2). The iPSC hepatocytes had active cytochrome p450, lipid and carbohydrate metabolic pathways (FIGS. 2 and 3). The inventors observed that cytochrome p450 genes and transporters were differentially expressed in iHeps, fetal hepatocytes and HepG2. This is not surprising given the variation in CYP gene induction kinetics and CYP polymorphisms (pharmacogenetics) in individuals of different genetic background that have been shown to affect drug metabolism and clearance [44, 45, 62, 63]. Establishing a repertoire of well characterized iPSC lines from individuals of diverse genetic background and/or collection of iPSC lines with genetically engineered CYP genes can be useful for the treatment of a range of liver diseases due to drug overdose or altered CYP metabolic activity.

In general, hepatic cells derived from pluripotent stem cells using current differentiation protocols are immature as demonstrated by presence of alpha fetoprotein (FIG. 2C). In adult liver, AFP expression is below detectable. The inventors observed that the iHeps cultured in the hollow fiber cartridge with continuous flow exhibited an approximately two order of magnitude reduction in AFP expression with concomitant increase in mature liver marker (FIG. 6). This finding has great practical value as mature hepatocytes are critical for assessing the toxicity and pharmacokinetic properties of drug compounds during preclinical development phase. Moreover, iHeps matured under continuous flow conditions can be a valuable cell source for cell therapy application towards inherited liver metabolic disorders.

In some embodiments, a hollow fiber capillary design is utilized for its high surface area and resemblance to natural blood capillary liver microarchitecture for nutrient and gas exchange. This design has been used in other liver support devices such as extracorporeal liver assist device (ELAD), and HepatAssist liver support system [11, 26]. Additional design features such as cartridge having layers of polysulfone membrane sheets where alternate layers filled with cells or blood/plasma flow can be considered. Another design consideration is improving the throughput. In some embodiments, a single cartridge closed circuit system may be utilized. For testing multiple growth conditions, animal experiments, and human clinical studies, a multi-cartridge system can be useful. Integrated multi-cartridge systems can allow for parallel production, better quality control, and uniformity among bioartificial liver devices.

In some embodiments, the inventors utilize dextran microcarrier beads for growing iHeps in suspension culture (FIG. 4). An advantage of using microcarrier suspension, in addition to the potential for scale culturing, is that iHep-beads can be easily transferred between culture modules without having the stress of an enzymatic dissociation process such as trypsinization. The iHep microcarrier culture approach can provide additional benefit such as minimizing their clogging of pores in the hollow fiber by cells. If the cells are directly attaching and expanding on the surface of hollow fiber, there is a high possibility of pore obstruction which can prevent exchange of gas, metabolites and nutrient in and out of the fiber capillaries.

The BAL devices disclosed herein may be further developed and validated for rescuing disease specific decompensated liver functions. In acute liver failure due to acetaminophen (APAP; paracetamol) poisoning, the liver cannot detoxify ammonia and secrete blood coagulation proteins such as prothrombin which results in increased intracranial pressure and bleeding [64, 65]. For this purpose, APAP metabolism and lethal concentration, and ammonia detoxification by iHeps need to be assessed. Moreover, unbiased analysis of metabolites and proteins that are secreted by iHeps can be conducted using a mass spectrometer. The secreted liver proteins and metabolites can be used as biomarkers for defining the therapeutic use window of an iHepBAL in testing the efficacy in an animal and human.

For clinical applications, the iHepBAL device has to reconstitute the function of the damaged liver and improve the health condition of the patient. In vivo preclinical studies in the rat and pig model systems of liver failure will provide insights into safety and efficacy of an iHepBAL device. For preclinical testing, anhepatic rat and acetaminophen toxicity in pig testing can be used [66, 67]. Based on plasma proteins, albumin and apoB, secretion data (FIG. 6), the iHeps can be cultured for 2 days in the cartridge after loading and subsequently be used for liver support purpose for in vivo testing. In some embodiments, a 70 cm² minicartridge may be utilized. For testing the efficacy of an iHepBAL in a large animal setting, a larger HFB cartridge (6000 cm²) with a loading density of over 1×10⁹ (one billion) cells may be utilized. Scaling up of iHep production is a key component, which can be accomplished by differentiating and expanding cells on microcarriers in a suspension culture. FIG. 7 shows a concept outline of iHep based extracorporeal closed circuit BAL system for supporting a liver failure patient, where the patient's plasma will be circulated through the iHep loaded cartridge for detoxification and plasma protein reconstitution.

### CONCLUSIONS

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the methods of deriving insulin-producing cells from pluripotent stem cells, preparing, isolating, or modifying cells used in the described differentiation techniques, derivation of insulin-producing cell lines from the aforementioned techniques, treatment of diseases and/or conditions that relate to the teachings of the invention, techniques and composition and use of solutions used therein, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

### REFERENCES

1 Centers for Disease Control and Prevention. Hepatitis Awareness. Available at http://www.cdc.gov/Features/dsHepatitisAwareness/. Accessed Jul. 16, 2014.

2 Hoofnagle J H, Carithers R L, Jr., Shapiro C, et al. Fulminant hepatic failure: summary of a workshop. Hepatology 1995: 21(1): 240252.

3 Bernal W, Auzinger G, Dhawan A, et al. Acute liver failure. Lancet 2010: 376(9736):190-201.

4 Carpentier B, Gautier A., Legallais C. Artificial and bioartificial liver devices: present and future. Gut 2009: 58(12):16901702.

5 Ellis A J, Hughes R D, Wendon J A, et al. Pilot controlled trial of the extracorporeal liver assist device in acute liver failure. Hepatology 1996: 24(6):14461451.

6 MacDonald J M, Wolfe S P, Roy Chowdhury I, et al. Effect of flow configuration and membrane characteristics on membrane fouling in a novel multi-coaxial hollow fiber bioartificial liver. Ann N. Y. Acad. Sci. 2001: 944:334343.

7 Rocen M, Kieslichova E, Merta D, et al. The effect of Prometheus device on laboratory markers of inflammation and tissue regeneration in acute liver failure management. Transplant. Proc. 2010: 42(9):36063611.

8 Sussman N L, Chong M G, Koussayer T, et al. Reversal of fulminant hepatic failure using an extracorporeal liver assist device. Hepatology 1992: 16(1):6065.

9 Iwata H, Ueda Y. Pharmacokinetic considerations in development of a bioartificial liver. Clin. Pharmacokinet. 2004: 43(4):211225.

10 Demetriou A A. Hepatic assist devices. Panminerva Med. 2005: 47(1):3137.

11 Demetriou A A, Brown R S, Jr., Busuttil R W, et al. Prospective, randomized, multicenter, controlled trial of a bioartificial liver in treating acute liver failure. Ann. Surg. 2004:239(5):660667; discussion 667670.

12 Rozga J, Williams F, Ro M S, et al. Development of a bioartificial liver: properties and function of a hollow fiber module inoculated with liver cells. Hepatology 1993: 17(2):258 265.

13 Kiley J E, Welch H F, Pender J C et al. Removal of blood ammonia by hemodialysis. Proc. Soc. Exp. Biol. Med. 1956: 91(3):489490.

14 Parbhoo S P, James I M, Ajdukiewicz A, et al. Extracorporeal pig liver perfusion in treatment of hepatic coma due to fulminant hepatitis. Lancet 1971: 1(7701):659665.

15 Sugiyama N, Rozga J, Demetriou A A. Biological extracorporeal liver assist devices: A minireview. Transplant Rev 2001: 15(3):129141.

16 Hui T, Rozga J, Demetriou A A. Bioartificial liver support. J. Hepatobiliary Pancreat. Surg. 2001: 8(1):115.

17 Watanabe F D, Mullon C J, Hewitt W R, et al. Clinical experience with a bioartificial liver in the treatment of severe liver failure. A phase I clinical trial. Ann. Surg. 1997: 225(5):484491; discussion 491484.

18 Laleman W, Wilmer A, Evenepoel P, et al. Effect of the molecular adsorbent recirculating system and Prometheus devices on systemic haemodynamics and vasoactive agents in patients with acute or chronic alcoholic liver failure. Crit. care 2006: 10(4):R108.

19 Ichai P, Samuel D. Treatment of patients with hepatic failure: the difficult place of liver support systems. J. Hepatol 2004: 41(4):694695.

20 Rifai K, Ernst T, Kretschmer U, et al. Prometheus: a new extracorporeal system for the treatment of liver failure. J. Hepatol 2003: 39(6):984990.

21 Matsubara S, Okabe K, Ouchi K, et al. Continuous removal of middle molecules by hemofiltration in patients with acute liver failure. Crit. Care Med. 1990: 18(12):13311338.

22 O'Grady J G, Gimson A E, O'Brien C J, et al. Controlled trials of charcoal hemoperfusion and prognostic factors in fulminant hepatic failure. Gastroenterology 1988: 94(5 Pt. 1):1186 1192.

23 Nyberg S L, Remmel R P, Mann H J, et al. Primary hepatocytes outperform Hep G2 cells as the source of biotransformation functions in a bioartificial liver. Ann. Surg. 1994: 220(1):5967.

24 Hughes R D, Nicolaou N, Langley P G et al. Plasma cytokine levels and coagulation and complement activation during use of the extracorporeal liver assist device in acute liver failure. Artif. Organs 1998: 22(10):854858.

25 Moussy Y. Convective flow through a hollow fiber bioartificial liver. Artif. Organs 2003: 27(11):10411049.

26 Sussman N L, Kelly J H. Improved liver function following treatment with an extracorporeal liver assist device. Artif. Organs 1993: 17(1):2730.

27 Rozga J, Podesta L, LePage E et al. A bioartificial liver to treat severe acute liver failure. Ann. Surg. 1994: 219(5):538544; discussion 544536.

28 Wang L, Sun J, Li L et al. Comparison of porcine hepatocytes with human hepatoma (C3A) cells for use in a bioartificial liver support system. Cell Transplant 1998: 7(5):459468.

29 Patience C, Takeuchi Y, Weiss R A. Infection of human cells by an endogenous retrovirus of pigs. Nat Med 1997: 3(3):282286.

30 Roberts E A, Letarte M, Squire J et al. Characterization of human hepatocyte lines derived from normal liver tissue. Hepatology 1994: 19(6):13901399.

31 AlNedawi K, Meehan B, Micallef J et al. Intercellular transfer of the oncogenic receptor EGFRvIII by microvesicles derived from tumour cells. Nat. Cell. Biol. 2008: 10(5):619624.

32 Rak J. Microparticles in cancer. Semin. Thromb. Hemost. 2010: 36(8):888906.

33 Sareen D, Ebert A D, Heins B M et al. Inhibition of apoptosis blocks human motor neuron cell death in a stem cell model of spinal muscular atrophy. PloS one 2012: 7(6):e39113.

34 Rashid S T, Corbineau S, Hannan N J et al. Modeling inherited metabolic disorders of the liver using human induced pluripotent stem cells. J. Clin. Invest. 2010: 120(9):31273136.

35 Basma H, SotoGutierrez A, Yannam G R et al. Differentiation and transplantation of human embryonic stem cell derived hepatocytes. Gastroenterology 2009: 136(3):990999.

36 Chen Y F, Tseng C Y, Wang H W, et al. Rapid generation of mature hepatocyte like cells from human induced pluripotent stem cells by an efficient three step protocol. Hepatology 2012: 55(4):11931203.

37 Cheng X, Ying L, Lu L et al. Self-renewing endodermal progenitor lines generated from human pluripotent stem cells. Cell. Stem Cell 2012: 10(4):371384.

38 McGrath K E, Koniski A D, Maltby K M, et al. Embryonic expression and function of the chemokine SDF1 and its receptor, CXCR4. Dev. Biol. 1999: 213(2):442456.

39 Nair S, Schilling T F. Chemokine signaling controls endodermal migration during zebrafish gastrulation. Science 2008: 322(5898):8992.

40 Tam P P, Kanai Azuma M, Kanai Y. Early endoderm development in vertebrates: lineage differentiation and morphogenetic function. Curr. Opin. Genet. Dev. 2003: 13(4):393400.

41 Parker A J, Houston J B. Rate limiting steps in hepatic drug clearance: comparison of hepatocellular uptake and metabolism with microsomal metabolism of saquinavir, nelfinavir, and ritonavir. Drug Metab. Dispos. 2008: 36(7):13751384.

42 Lau Y Y, Sapidou E, Cui X, et al. Development of a novel in vitro model to predict hepatic clearance using fresh, cryopreserved, and sandwich cultured hepatocytes. Drug Metab. Dispos. 2002: 30(12):14461454.

43 McLean A E. Effect of hexane and carbon tetrachloride on microsomal cytochrome (P450). Biochem. Pharmacol. 1967: 16(10):20302033.

44 Iyer L, Ratain M J. Pharmacogenetics and cancer chemotherapy. Eur. J. Cancer 1998: 34(10):14931499.

45 Guengerich F P. Cytochrome P450: what have we learned and what are the future issues? Drug Metab. Rev. 2004: 36(2):159197.

46 Monostory K, Dvorak Z. Steroid regulation of drug metabolizing cytochromes P450. Curr. Drug Metab. 2011: 12(2):154172.

47 Zimniak P, Waxman D J. Liver Cytochrome P450 Metabolism of Endogenous Steroid Hormones, Bile Acids, and Fatty Acids. In: Cytochrome P450. Springer Berlin Heidelberg, 1993: 123144.

48 Sengupta S, Johnson B P, Swanson S A, et al. Aggregate Culture of Human Embryonic Stem Cell Derived Hepatocytes in Suspension are an Improved In Vitro Model for Drug Metabolism and Toxicity Testing. Toxicol. Sci.2014.

49 Du Y, Wang J, Jia J, et al. Human hepatocytes with drug metabolic function induced from fibroblasts by lineage reprogramming. Cell stem cell 2014: 14(3):394403.

50 Lavon N, Yanuka O, Benvenisty N. Differentiation and isolation of hepatocyte like cells from human embryonic stem cells. Differentiation 2004: 72(5):230238.

51 Ruhnke M, Ungefroren H, Nussler A, et al. Differentiation of in vitro modified human peripheral blood monocytes into hepatocyte like and pancreatic islet like cells. Gastroenterology 2005: 128(7):17741786.

52 Hay D C, Fletcher J, Payne C, et al. Highly efficient differentiation of hESCs to functional hepatic endoderm requires Activin A and Wnt3a signaling. Proc. Natl. Acad. Sci. U. S. A. 2008:105(34):12301-12306

53 Hay D C, Zhao D, Fletcher J et al. Efficient differentiation of hepatocytes from human embryonic stem cells exhibiting markers recapitulating liver development in vivo. Stem cells 2008: 26(4):894902.

54 Campard D, Lysy P A, Najimi M, Sokal E M. Native umbilical cord matrix stem cells express hepatic markers and differentiate into hepatocyte like cells. Gastroenterology 2008: 134(3):833848.

55 Cai J, Zhao Y, Liu Y, et al. Directed differentiation of human embryonic stem cells into functional hepatic cells. Hepatology 2007: 45(5):12291239.

56 Aurich H, Sgodda M, Kaltwasser P, et al. Hepatocyte differentiation of mesenchymal stem cells from human adipose tissue in vitro promotes hepatic integration in vivo. Gut 2009: 58(4):570581.

57 Schwartz R E, Linehan J L, Painschab M S, et al. Defined conditions for development of functional hepatic cells from human embryonic stem cells. Stem Cells Dev. 2005: 14(6):643 655.

58 GouonEvans V, Boussemart L, Gadue P, et al. BMP4 is required for hepatic specification of mouse embryonic stem cell derived definitive endoderm. Nat. Biotechnol. 2006: 24(11):14021411.

59 Touboul T, Hannan N R, Corbineau S, et al. Generation of functional hepatocytes from human embryonic stem cells under chemically defined conditions that recapitulate liver development. Hepatology 2010: 51(5):17541765.

60 Duan Y, Ma X, Zou W, et al. Differentiation and characterization of metabolically functioning hepatocytes from human embryonic stem cells. Stem cells 2010: 28(4):674686.

61 Agarwal S, Holton K L, Lanza R. Efficient differentiation of functional hepatocytes from human embryonic stem cells. Stem cells 2008: 26(5):11171127.

62 Wojtczak A, Wojtczak M, Skretkowicz J. The relationship between plasma concentration of metoprolol and CYP2D6 genotype in patients with ischemic heart disease. Pharmacol. Rep 2014: 66(3):511514.

63 Bray J, Sludden J, Griffin M J, et al. Influence of pharmacogenetics on response and toxicity in breast cancer patients treated with doxorubicin and cyclophosphamide. Br J Cancer 2010: 102(6):10031009.

64 James L P, Simpson P M, Farrar H C, et al. Cytokines and toxicity in acetaminophen Overdose J. Clin. Pharmacol 2005: 45(10):11651171.

65 Prescott L F, Roscoe P, Wright N, et al. Plasma paracetamol half-life and hepatic necrosis in patients with paracetamol over-dosage. Lancet 1971: 1(7698):519522.

66 Arkadopoulos N, Lilja H, Suh K S, et al. Intrasplenic transplantation of allogeneic hepatocytes prolongs survival in anhepatic rats. Hepatology 1998: 28(5):13651370.

67 Lee K C, Palacios Jimenez C, Alibhai H, et al. A reproducible, clinically relevant, intensively managed, pig model of acute liver failure for testing of therapies aimed to prolong survival. Liver Int. 2013: 33(4):544551.

1. A bioreactor module for a bioartificial liver device, the module comprising:
a continuous flow bioreactor; and
a quantity of induced pluripotent stem cell derived hepatocytes (i-Heps) that are fixed to microcarrier beads in the extracapillary space of the continuous flow bioreactor.

2. The bioreactor module of claim 1, wherein the continuous flow bioreactor is a hollow-fiber bioreactor.

3. The bioreactor module of claim 1, wherein the continuous flow bioreactor is in fluid communication with a patient's plasma.

4. The bioreactor module of claim 1, wherein the microcarriers have a pore size of less than 1 μm.

5. The bioreactor module of claim 1, wherein the microcarriers are coated with laminin.

6. The bioreactor module of claim 1, wherein the microcarriers have a particle size from 60 to 87 μm.

7. The bioreactor module of claim 1, wherein the microcarriers comprise dextran.

8. The bioreactor module of claim 1, wherein the bioreactor has hollow fibers with 0.21 μm pores.

9. The bioreactor module of claim 3, wherein an ultrafiltrate generator is in fluid communication with a patient's blood.

10. A method of using a bioartificial liver device in order to support, enhance, or replace liver function of a patient, the method comprising:
(a) removing plasma from a patient's blood stream;
(b) pumping the plasma through a bioartificial liver module, wherein the bioartificial liver module includes a quantity of induced pluripotent stem cell derived hepatocytes (iHeps) that are fixed to microcarrier beads in the extracapillary space of the bioartificial liver module; and
(c) returning the plasma to a patient's blood stream.

11. The method of claim 10, wherein the iHeps are cultured in a microcarrier suspension culture prior to introduction into the extracapillary space of the bioartificial liver module.

12. The method of claim 11, wherein the iHeps are matured in a continuous flow bioreactor while fixed to microcarrier beads prior to pumping the plasma through the bioartificial liver module.

13. The method of claim 10, wherein the microcarriers have a pore size of less than 1 μm.

14. The method of claim 10, wherein the microcarriers are coated with laminin.

15. The method of claim 10, wherein the microcarriers have a particle size from 60 to 87 μm.

16. The method of claim 10, wherein the microcarriers comprise dextran.

17. The method of claim 10, wherein the bioreactor has hollow fibers with 0.21 μm pores.

18. A method of differentiating a human pluripotent stem cell into a cell capable of hepatic function comprising:
(a) providing a quantity of induced pluripotent stem cells (pSCs);
(b) culturing the pSCs in the presence of at least one differentiation agent, wherein the at least one differentiation agent is capable of differentiating the pSCs into an induced pSC-derived hepatocyte (iHep);
(c) harvesting the iHep and pre-culturing the iHep on microcarrier beads; and
(d) further culturing the iHep on the microcarrier beads in a continuous flow bioreactor to form a mature hepatocyte from the iHep.

19. The method of claim 18, wherein the continuous flow bioreactor is a hollow fiber bioreactor (HFB).

20. The method of claim 18, wherein the microcarriers have a pore size of less than 1 μm.

21. The method of claim 18, wherein the iHep is further cultured in the continuous flow bioreactor until the expression of AFP is reduced 50 fold.

22. The method of claim 18, wherein the step (c) is performed between day 16-day 21 of a differentiation protocol.

23. The method of claim 18, wherein the step (c) is performed on day 19 of a differentiation protocol.

24. The method of claim 18, wherein the microcarriers are coated with laminin.

25. The method of claim 18, wherein the bioreactor has hollow fibers with 0.21 μm pores.

26. The method of claim 18, wherein the microcarriers have a particle size from 60 to 87 μm.

27. The method of claim 18, wherein the microcarriers comprise dextran.

28. The method of claim 18, further comprising:
(c) culturing the pSCs in the presence of at least one second differentiation agent comprising Activin A;
(d) culturing of the pSCs in the presence of at least one third differentiation agent comprising VEGF; and
(e) culturing the pSCs in the presence of at least one fourth differentiation agent comprising EGF, TGF-α, and dexamethasone, bFGF, and BMP4.

29. The method of claim 28, wherein the cells are further cultured in the presence of at least a first maturation agent comprising: HGF, dexamethasone, and oncostatin M.

30. A cell line, comprising one or cells produced by the method of claim 18.

31. A method of differentiating a human pluripotent stem cell into a cell capable of hepatic function comprising:
(a) providing a quantity of human pluripotent stem cells (pSCs);
(b) culturing the pSCs in the presence of at least one differentiation agent, wherein the at least one differentiation agent is capable of differentiating the pSCs into an induced pSC-derived hepatocyte (iHep); and
(c) wherein the culturing is at least partially performed in a continuous flow bioreactor with the pSCs or cells differentiated from the pSCs adhered to microcarrier beads.

32. The method of claim 31, wherein the continuous flow bioreactor is a hollow fiber bioreactor (HFB).

33. The method of claim 31, wherein the microcarriers have a pore size of less than 1 μm.

34. The method of claim 31, wherein the iHep is further cultured in the continuous flow bioreactor until the expression of AFP is reduced 50 fold.

35. The method of claim 31, wherein step (c) is performed between day 16-day 21 of a differentiation protocol.

36. The method of claim 31, wherein step (b) is performed between day 12-day 21 of a differentiation protocol.

37. The method of claim 31, wherein step (c) is performed on day 19 of a differentiation protocol.

38. The method of claim 31, wherein step (c) is performed for an entire differentiation protocol.

39. The method of claim 31, wherein the microcarriers are coated with laminin.

40. The method of claim 31, wherein the bioreactor has hollow fibers with 0.21 μm pores.

41. The method of claim 31, wherein the microcarriers have a particle size of 60-87 μm.

42. The method of claim 31, wherein the microcarriers are dextran microcarrier beads.
